(19) United States
(12) Patent Application Publication
(10) Pub. No.: US 2014/0046140 A1
(43) Pub. Date: Feb. 13, 2014

(54) CERCLAGE SUTURE REMOVAL DEVICE

(71) Applicant: Eastern Virginia Medical School, Norfolk, VA (US)

(72) Inventor: Alfred Z. ABUHAMAD, Norfolk, VA (US)

(73) Assignee: Eastern Virginia Medical School, Norfolk, VA (US)

(21) Appl. No.: 14/056,871

(22) Filed: Oct. 17, 2013

Related U.S. Application Data
(63) Continuation-in-part of application No. PCT/US2012/034000, filed on Apr. 18, 2012.
(60) Provisional application No. 61/476,625, filed on Apr. 18, 2011.
(60) Provisional application No. 61/784,350, filed on Mar. 14, 2013.

(51) Int. Cl. A61B 17/42; A61B 17/02

(52) U.S. Cl. CPC A61B 17/42; A61B 17/0218
USPC 600/245; 606/119; 600/235

(57) ABSTRACT

A device for removing a cerclage suture has a shaft sized and shaped to extend from a proximal end outside a vagina to a distal end near a cervix. The shaft also has a blunt end located at the distal end of the shaft and having a taper for allowing the blunt end to pass between a cerclage suture and cervical mucosa without cutting the mucosa and to move the suture away from the mucosa. The device includes a cutter having at least one blade at the distal end of the shaft. The cutter may be movable between an open non-cutting position and a closed cutting position for cutting the suture. Alternatively, the cutter may include one or more stationary blades. The device also may include a handle at the proximal end of the shaft that may comprise an actuator or may comprise a cylindrical or another suitable shape.

### RELATED APPLICATION INFORMATION

This application is a continuation-in-part of PCT Application Ser. No. PCT/US12/034000, filed on Apr. 18, 2012, which claims the benefit of U.S. Provisional Application Ser. No. 61/476,625, filed on Apr. 18, 2011, the contents of which are hereby incorporated-by-reference. This Application also claims the benefit of U.S. Provisional Application Ser. No. 61/784,350 filed on Mar. 14, 2013, the contents of which are hereby incorporated-by-reference.

### FIELD

This disclosure relates to a device for removing a cerclage suture.

### BACKGROUND

Cerclage placement is a common procedure performed in high risk pregnancies where a woman is at risk for pregnancy loss from incompetent cervix or a short cervix as identified on ultrasound examination. Cerclage placement in women with a short cervix can reduce preterm delivery. Cerclage placement is typically done in early second trimester and the procedure is performed in the hospital under regional anesthesia. The procedure involves placement of a suture around the cervix in a purse-string fashion which provides compression of the cervix and prevents the premature opening of the cervix.

The suture itself is typically removed at about thirty-seven weeks gestation. This length of time between placement of the suture and time for its removal can cause mucosal swelling surrounding the suture and thus makes its removal difficult. Cerclage removal typically involves trying to identify the loop of the suture and using scissors to remove it under direct visualization. This can be a difficult procedure as the free loop is buried under the cervical mucosa and can be difficult to identify.

Due to the overgrowth of the cervical mucosa and the difficulty of cerclage removal using currently available instruments, in many occasions, cerclage removal is performed in the inpatient setting under regional anesthesia. Having a device that can assist in cerclage removal will therefore simplify the procedure and allow for the majority of cerclage removals to be performed in the outpatient settings with minimal local anesthesia.

### SUMMARY

This disclosure describes a device for removing a cerclage. In one aspect, the device has a shaft that has a cross sectional area and a cross sectional shape, and is sized and shaped to extend from a proximal end outside a vagina to a distal end near a cervix. The shaft also has a blunt end located at the distal end of the shaft and having a taper for allowing the blunt end to pass between a cerclage suture and cervical mucosa without cutting the mucosa and to move the suture away from the mucosa. The device also comprises a cutter having at least one blade at the distal end of the shaft. The cutter may be movable between an open non-cutting position and a closed cutting position for cutting the suture. Alternatively, the cutter may include one or more stationary blades. The device also may comprise a handle at the proximal end of the shaft. The handle may comprise an actuator for causing the cutter to be moved from the open position to the closed position to cut the suture. Alternatively, the handle may comprise a cylindrical shape, or another suitable shape for allowing an operator to grip the distal end of the shaft. The device may include a lip on the blunt end defining a recess to hold the suture and to prevent the suture from slipping off of the blunt end of the shaft before the suture is cut. The device may further comprise a recess on the blunt end of the shaft for holding the suture. In some embodiments, the surface of the blunt end that passes between the cerclage suture and cervical mucosa does not include a blade.

In some embodiments, the cutter includes a first relatively movable part and a second relatively stationary part, the cutter including a blade on one of the first and second parts. In other embodiments, the blade is on the movable part and is retractable. The cutter may include a first part that is movable relative to the shaft, and a pivoting pin at the distal end of the shaft, the pivoting pin allowing the movable part of the cutter to rotate about the pivoting pin to the closed cutting position when actuated. The cutter may include a movable portion that is recessed within the shaft and is moved away from the shaft to the closed cutting position when the cutter is actuated. The movable portion may return to a prior location in the distal end of the shaft after the suture is cut. The cutter may be spring-biased to an open position. The cutter may include a blade on the movable portion. Alternatively, the cutter may include a blade that is not on the movable portion.

In some embodiments, the actuator may be coupled to the proximal end of the shaft for allowing activation of the cutter when the distal end of the shaft is near the cervix. In other embodiments, the actuator may include a rod having a distal portion coaxial with the shaft for engaging the cutter to cause the cutter to move to the closed cutting position. The distal portion of the rod may include an inclined wedge that slides under the cutter and moves the cutter into the closed cutting position when actuated. The device may include a light source for visualization of the suture. In some embodiments, a speculum is inserted into the vagina to hold open a vaginal canal.

This disclosure also includes a method for removing a cerclage. In this method, a shaft having a blunt end is inserted into a patient's vagina. The blunt end is positioned under a cervical suture to cause the suture to move away from a cervical mucosa, wherein the blunt end that passes under the cervical suture does not have a cutting blade. A suture is cut either by actuating a retractable cutter, by pulling the device toward the operator, thereby causing the blade of the cutter to cut the suture, or by otherwise moving the cutter relative to the suture. Then, the shaft is withdrawn from the patient's vagina. In some embodiments, prior to the inserting the shaft, a speculum is inserted into the patient's vagina, then the shaft is inserted through the speculum. Additionally, in some embodiments, the actuator causes a first portion of the cutter that is movable relative to the shaft to pivot about a pivot point at a distal end of the shaft.

### BRIEF DESCRIPTION OF THE FIGURES

FIG. 1 is a cross sectional view of a baby in the uterus before cerclage.

FIG. 2 is a cross sectional view of a baby in the uterus with a suture around the cervix.

FIG. 1A is a cross sectional view of the suture pathway.

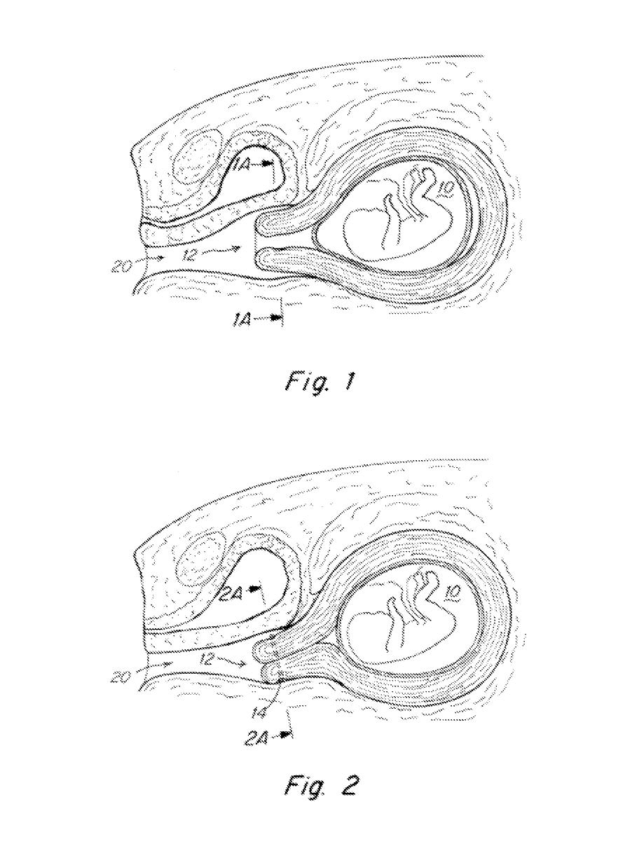

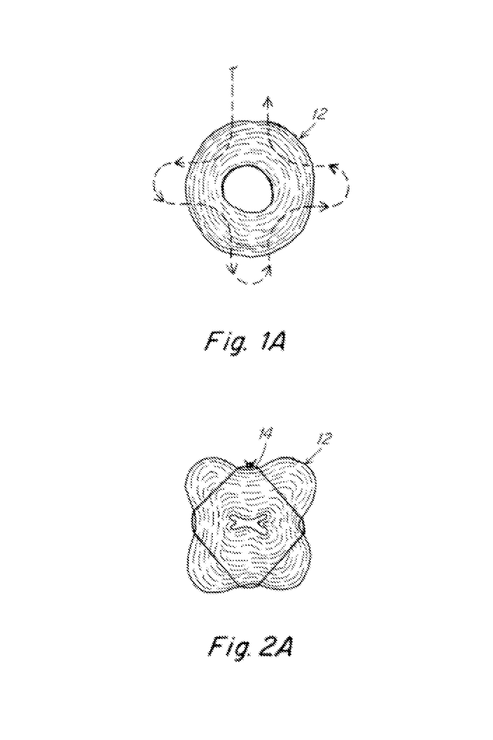

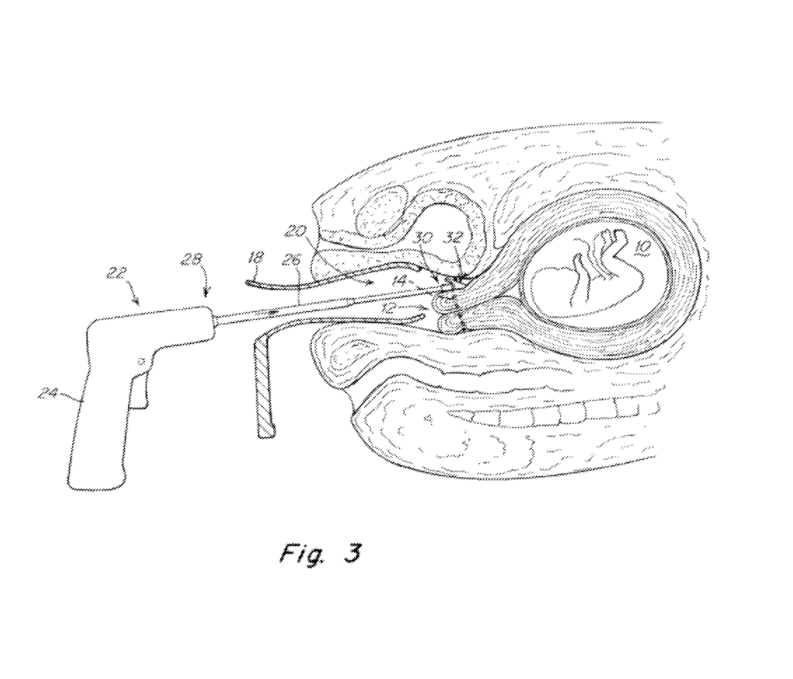

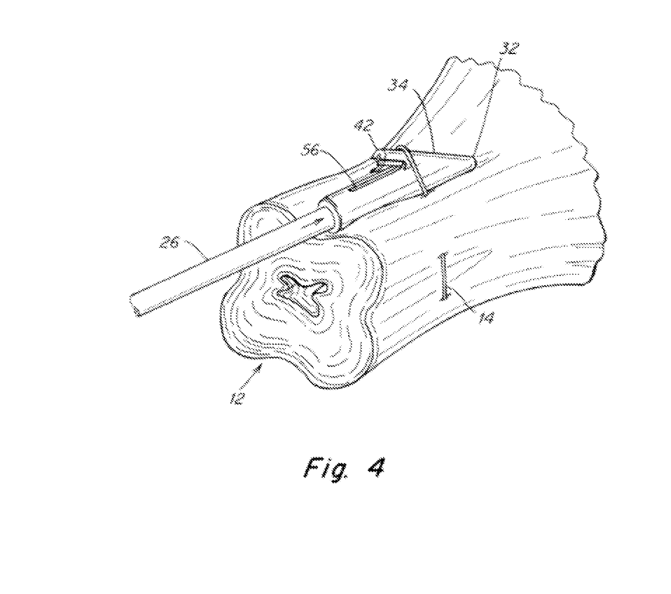

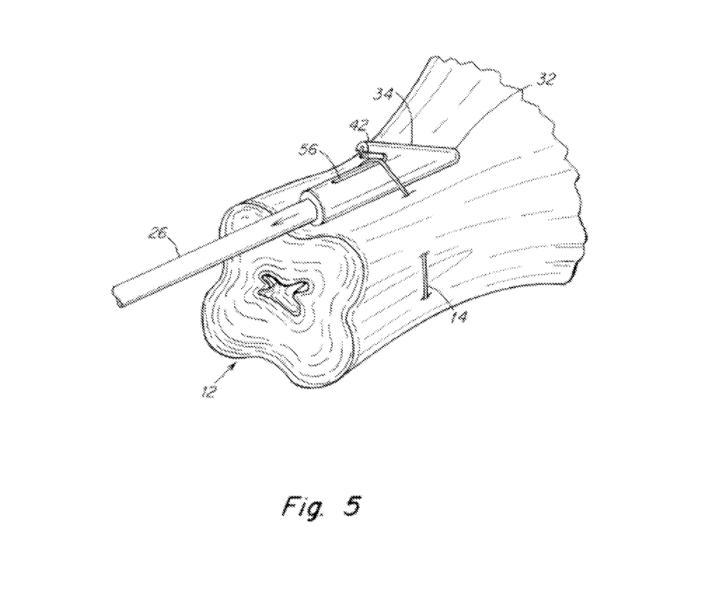

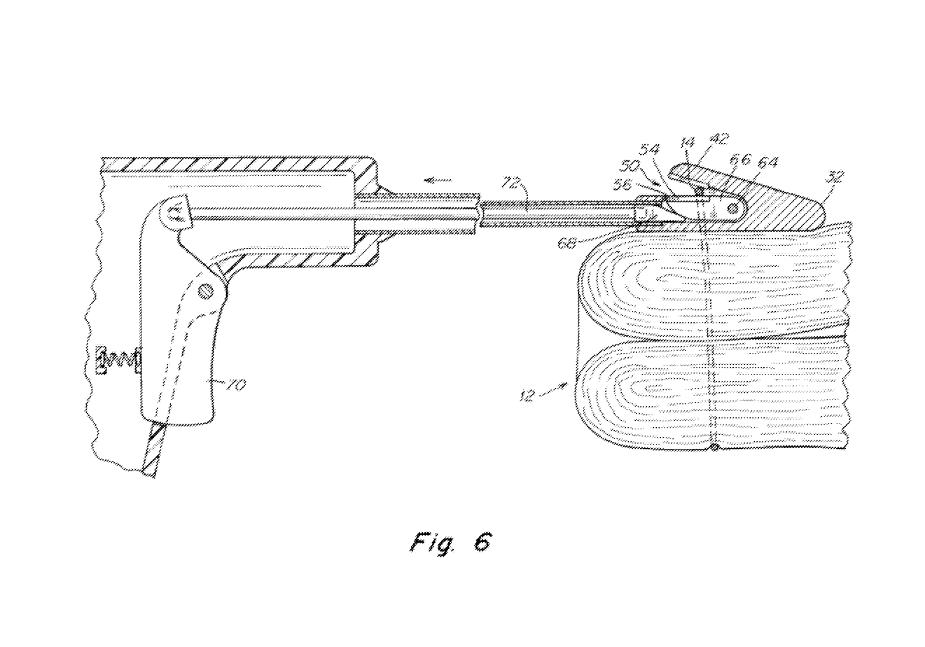

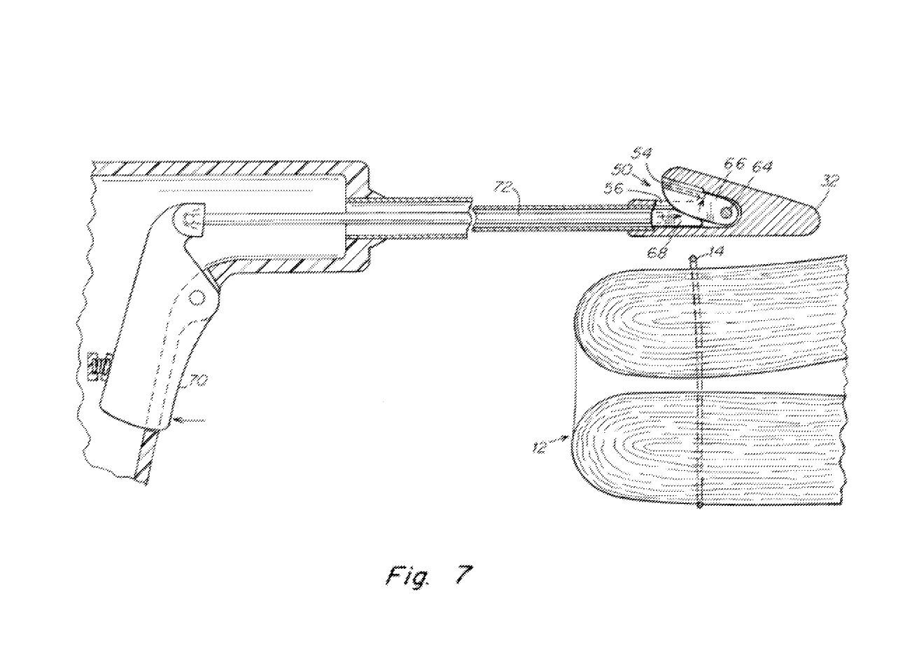

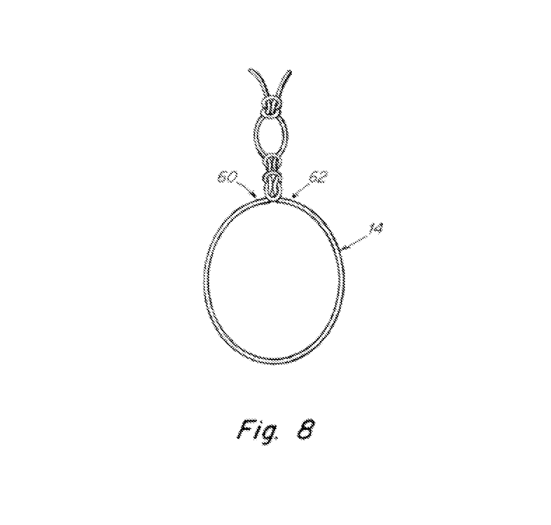

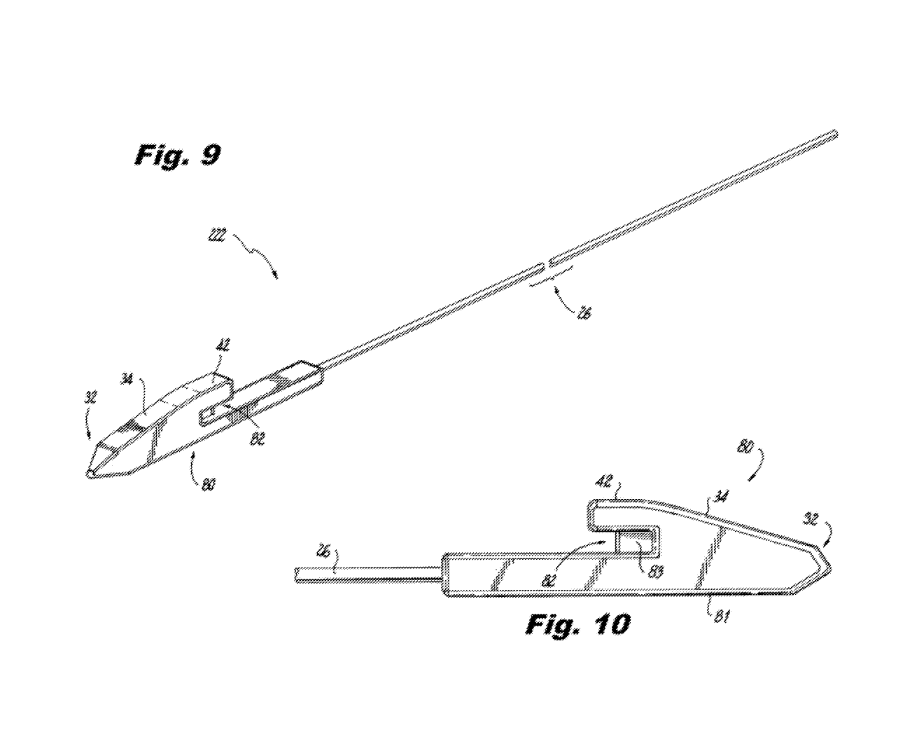

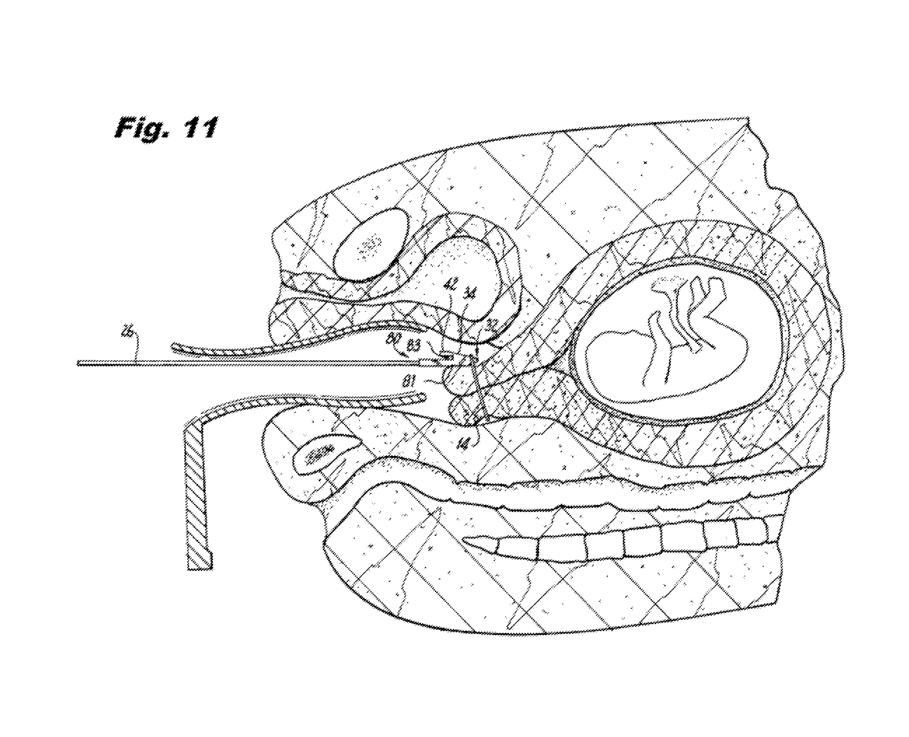

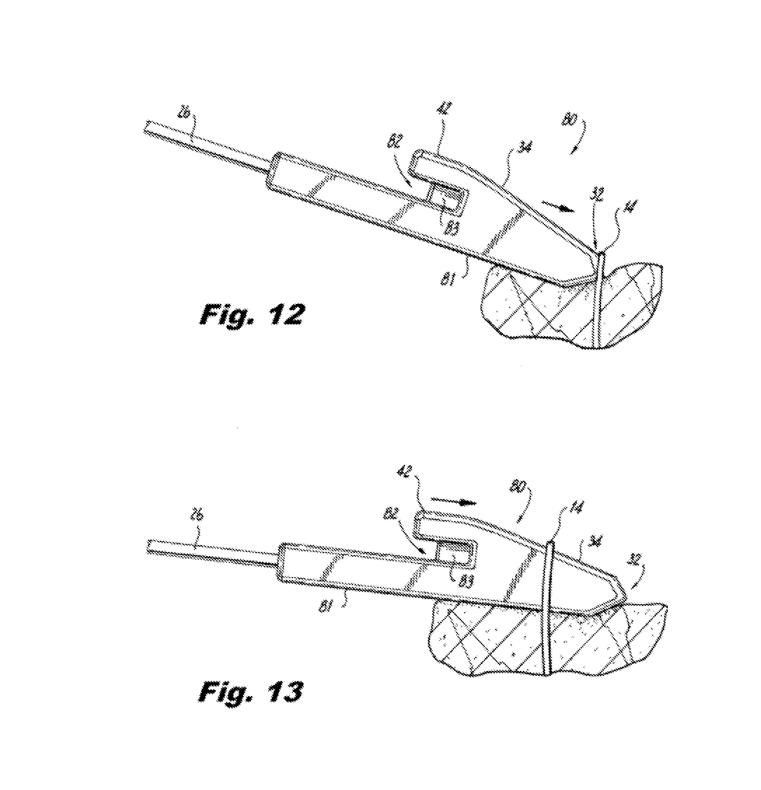

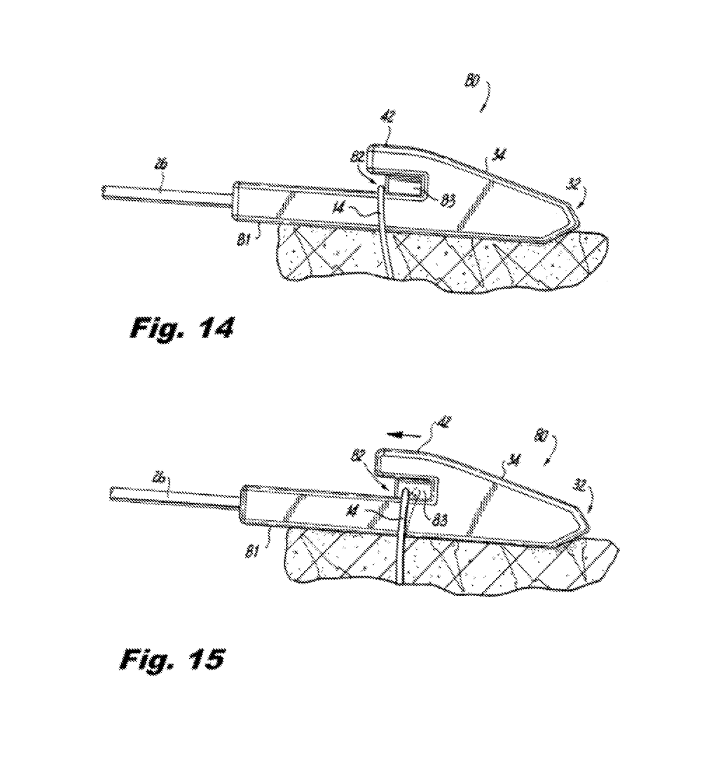

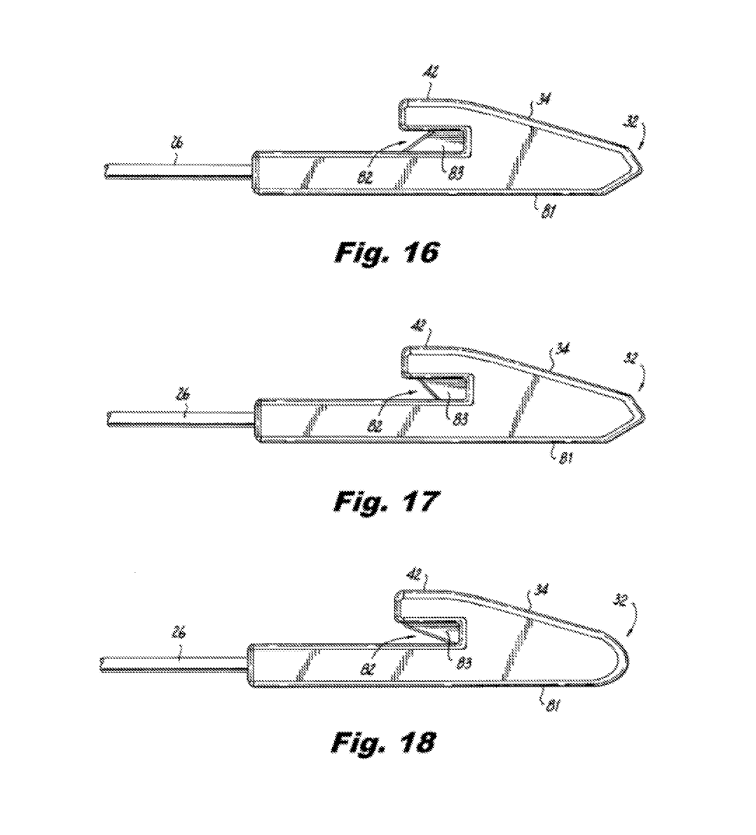

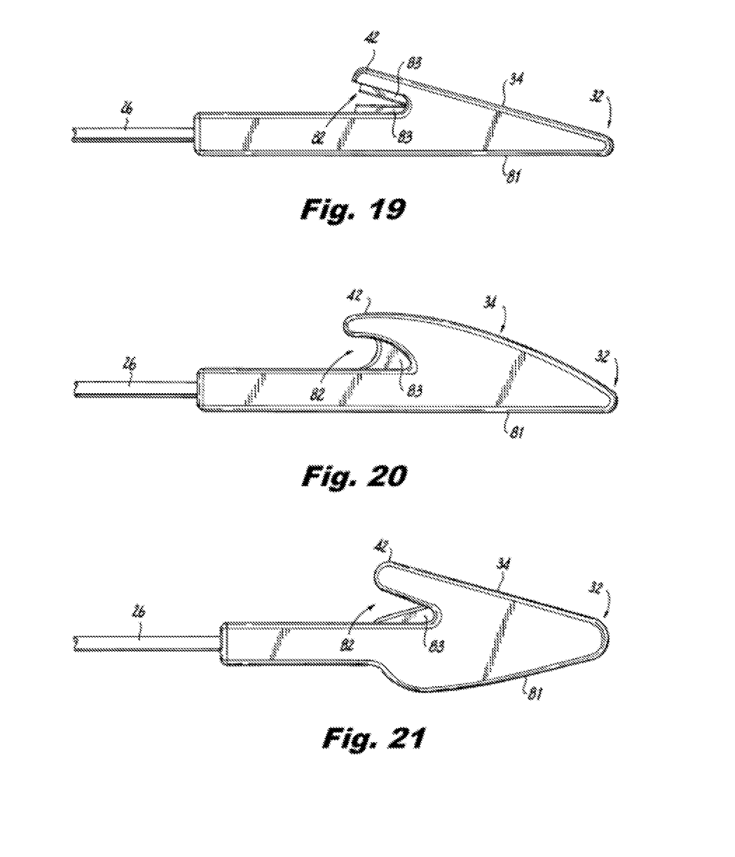

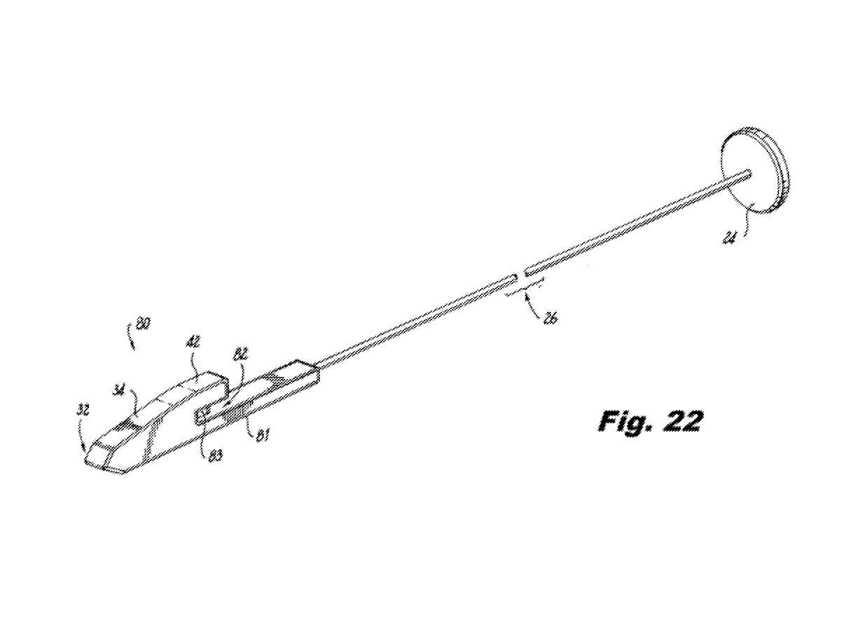

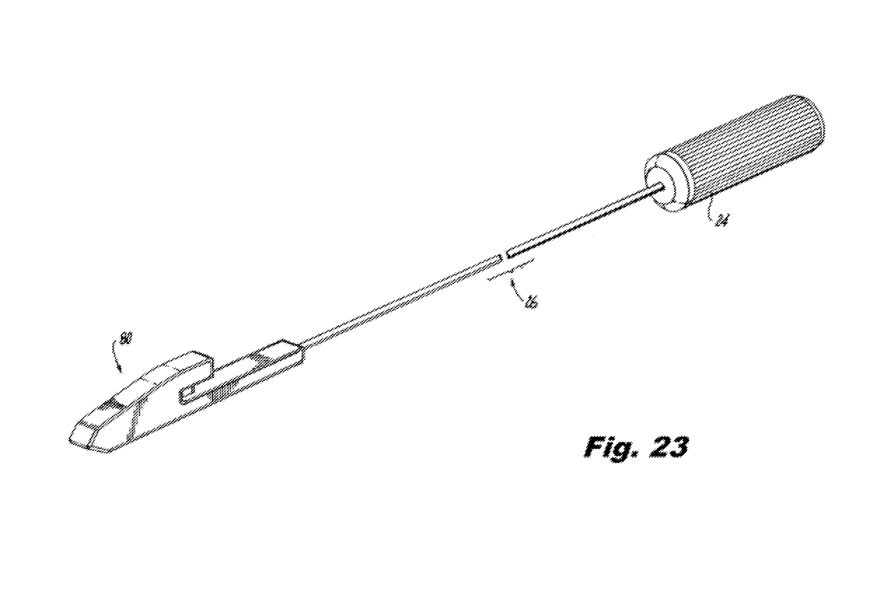

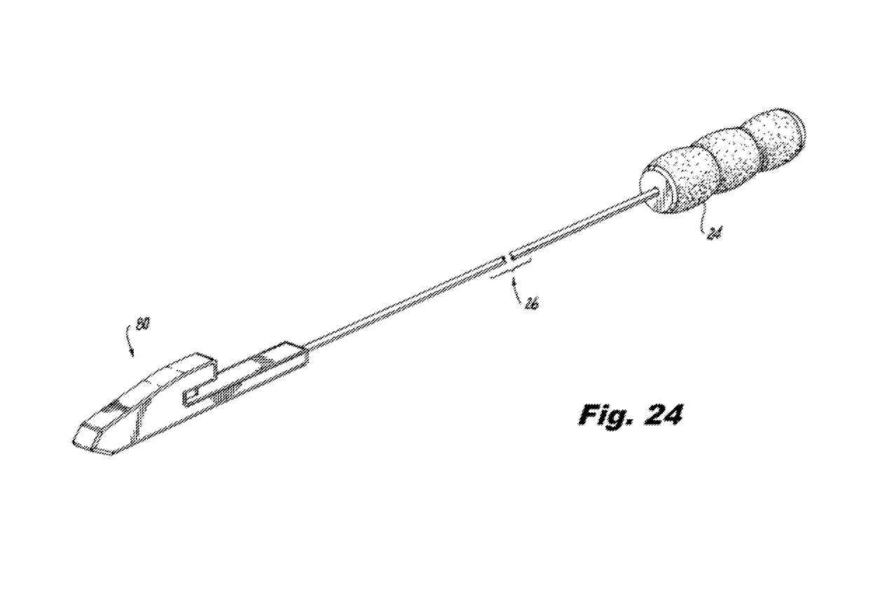

FIG. 2A is a cross sectional view of a suture around the cervix.

FIG. 3 is a cross sectional view of a shaft extending into the vaginal canal to the cervix.

FIG. 4 is a perspective view of the blunt end of the shaft under the cervical suture.

FIG. 5 is a perspective view of the tip of the shaft in an open position under the cervical suture.

FIG. 6 is a cross sectional view of an embodiment of the shaft in an open position under the cervical suture.

FIG. 7 is a cross sectional view of an embodiment of the shaft in a closed position and cutting the cervical suture.

FIG. 8 is a view of a cerclage with cutting locations.

FIG. 9 is a perspective view of an embodiment of the device having a stationary cutting blade.

FIG. 10 is a side view of the embodiment of FIG. 9.

FIG. 11 is a side view of the embodiment of FIG. 9 under the cervical suture.

FIG. 12 is a side view of the embodiment of FIG. 9 where the blunt end of the shaft is lifting the cervical suture from the cervical mucosa.

FIG. 13 is a side view of the embodiment of FIG. 9 where the blunt end of the shaft is under the cerclage suture.

FIG. 14 is a side view of the embodiment of FIG. 9 where the cerclage suture is within the recess.

FIG. 15 is a side view of the embodiment of FIG. 9 moving with respect to the suture and cutting the cervical suture.

FIG. 16 is an alternative embodiment of the device having a head with a stationary blade.

FIG. 17 is an alternative embodiment of the device having a head with a stationary blade.

FIG. 18 is an alternative embodiment of the device having a head with a stationary blade.

FIG. 19 is an alternative embodiment of the device having a head with two stationary blades.

FIG. 20 is an alternative embodiment of the device having a head with a stationary blade.

FIG. 21 is alternative an embodiment of the device having a head with a stationary blade.

FIG. 22 is perspective view of an alternative embodiment of the device having a head, a shaft and a handle.

FIG. 23 is perspective view of an alternative embodiment of the device having a head, a shaft and a handle.

FIG. 24 is perspective view of an alternative embodiment of the device having a head, a shaft and a handle.

### DETAILED DESCRIPTION

This disclosure describes a device for removing a cerclage.

As shown in FIG. 1, a baby is shown in the uterus 10. There is no cerclage at the cervix 12. As used herein, the term “cerclage” includes a cervical stitch and a tracheloplasty in the cervix. The cervix 12 and the vaginal canal 20 are also shown.

Referring to FIG. 2, a baby is in the uterus and there is a cerclage 14 at the cervix 12.

FIGS. 1A and 2A are cross-sections of FIG. 2, at similar locations. The cerclage 14 is a cervical stitch through the cervical mucosa to close the cervix during pregnancy. FIG. 1A shows a pathway of a cervical suture. FIG. 2A shows a cross section of a completed cerclage. Other suitable stitch pathways for closing the cervix can also be used.

As shown in the embodiment in FIG. 3, a speculum 18 can be used to hold open the vaginal canal 20. A cerclage cutting device 22 has a handle 24 and an elongated shaft 26 with a proximal end 28 and a distal end 30. Shaft 26 is held with the handle which can be designed for left and right handed use, near proximal end 28. Shaft 26 can have a uniform cross sectional diameter, or as shown, can have sections, e.g., with a larger cross sectional diameter portion and a smaller cross sectional diameter portion. The shaft can include plastic, metal, or any other suitable material. The shaft may be hollow or solid. The shaft has a suitable length, diameter, size and shape, such that the distal end of the shaft is configured to extend into the vaginal canal to the cervix. The proximal end of the shaft is configured to remain outside of the vaginal canal.

The distal end of the shaft has a head including blunt end 32 for passing into the vaginal canal and tunneling under the cervical mucosa without cutting the cervical or vaginal tissue. The blunt end does not include a blade.

As shown more clearly in FIG. 4, the top side of the blunt end 32 may be tapered 34 so that the end can tunnel under the cerclage 14 and elevate the suture from the cervix 12. This elevation allows a clear visualization of the area where the cutting will be performed. In this embodiment, blunt end 32 has a curvature along its posterior aspect that allows the suture to be elevated, although other shapes typically tapered or curved can be used to move the suture away from the cervical tissue without cutting the cervical tissue. For example, as shown in FIGS. 9-20 and 22-24, the underside of the blunt end 81 may be parallel to the shaft 26. Alternatively, in the embodiment shown in FIG. 21, the underside of the blunt end 81 may be disposed at an angle relative to the shaft and may slope upward or downward relative to the shaft. As the shaft 26 is moved laterally under the cerclage 14, blunt end 32 can pass under the suture and lift the cerclage away from the cervical mucosa. This allows the cerclage to be cut without cutting the cervical or vaginal tissue. Although the top side of the blunt end 34 in FIG. 4 is tapered at a constant angle, other configurations are contemplated. For example, in the embodiment shown in FIG. 20, the top side of the blunt end 34 may be curved in shape, or the top side may include sections having varying degrees of taper.

In the embodiment shown in FIG. 5, the head of the shaft may include a lip 42. The lip 42 may extend from the blunt end 32 to define a recess in the head of the device. Alternatively the lip may be of a suitable configuration to catch the suture as it passes over the blunt end 32. Various embodiments of lip 42 are shown in FIGS. 4 and 9-24. For example, as shown in FIGS. 9-18 and 22-24, the lip 42 may extend parallel to the shaft 26, or the lip 42 may be curved as shown in FIG. 20. Other suitable configurations for holding the suture on the blunt end of the shaft may also be used. The recess 82 is configured to hold the suture and to prevent the suture from slipping off of the blunt end of the shaft before the suture is cut. As shown in FIGS. 4, 19, 21 and 22-24, the lip 42 may be disposed at the same angle as the taper of the top side of the blunt end 34 or may be of any suitable shape that holds the cerclage.

As shown in FIGS. 6 and 7, the lip 42 holds the suture in place so that it may be cut by a cutter 50 that can, in some embodiments, have a closed cutting position (FIG. 7) and an open non-cutting position (FIG. 6). The cutter is in an open position in the distal end before and after the cutter is actuated. In one embodiment, when the cutter is actuated, a cutter blade 54 moves through an opening 56 in the distal end of the shaft to a closed position. In the closed position, a blade extends through the distal end of the shaft and cuts the suture.

As shown in FIG. 8, there are two ideal locations 60, 62 for cutting the cerclage suture with the retractable cutter 50. The ideal locations 60, 62 for cutting the suture can be seen from the outside of the vaginal canal when the cerclage is elevated with the blunt end. FIG. 8 shows an example of cerclage without surrounding tissue.

Referring again to FIGS. 6 and 7, in one embodiment, the cutter includes a pin 64 that allows the cutter to rotate and pivot about the pin 64 to the closed cutting position when actuated. The blade of the cutter could be located on a relatively movable portion or a relatively stationary portion of the cutter. In this embodiment, a movable portion 66 of the cutter is lifted by a wedge 68 that moves horizontally under operation control and slides under and lifts the movable portion when the device is actuated because of the relationship of the surfaces. The movable portion returns to a prior location in the distal end of the shaft, e.g., with a spring.

Other suitable configurations for allowing the cutter to be actuated to cut the suture and to retract to prevent the cutter from cutting the cervical mucosa can also be used. As shown in FIGS. 9-24, the cutter may comprise one or more stationary blades 83 that are configured to cut a cerclage suture when the device is moved relative to the suture. The blade or blades may be located in the recess 82. For example, in the embodiment shown in FIG. 10, the cutter may comprise a single blade 83 disposed in the recess 82. In this embodiment, the cutting edge of the blade 83 is disposed substantially perpendicular to the shaft. As shown in FIG. 12, like in FIG. 4, the top side of the blunt end 32 may be tapered 34 so that the end can tunnel under the cerclage 14 and elevate the suture from the cervix. As shown in FIG. 13, the shaft 26 is moved laterally under the cerclage 14, blunt end 32 can pass under the suture and lift the cerclage away from the cervical mucosa. This allows the cerclage to be cut without cutting the cervical or vaginal tissue. As shown in FIG. 14 the lip holds the suture in place prior to cutting. Then, as shown in FIG. 15, the cerclage device is pulled toward the user such that the device moves relative to the suture and the blade 83 cuts the suture 14. Alternatively, the device may be configured such that the suture is cut when the device is pushed, lifted, or otherwise moved relative to the suture. As shown in FIGS. 10 and 16-21, the cutting edge of the blade 83 may be disposed at various angles with respect to the shaft or may be disposed in a variety of other suitable configurations. For example, as shown in FIGS. 16-18 and 21, the cutting edge of the blade may be at an acute angle or an obtuse angle with respect to the shaft. Alternatively, the blade may extend from the shaft and the cutting edge may be parallel to the shaft, or the blade may extend from the lip and the cutting edge may be parallel to the lip or the shaft. As shown in FIG. 19, more than one blade 83 may be disposed in the recess. For example, one blade may extend from the shaft, while a second blade may extend from the lip. The blades may meet in a V-shape at the distal end of the recess 82. Other suitable configurations for allowing the cutter to cut the suture without cutting the cervical mucosa can also be used.

The cutting edge of the blade may be smooth or serrated and is of sufficient sharpness to cut a cerclage suture. The height and width of the blade may vary depending on the gauge of the cerclage suture. For example, a thicker blade may be used to cut larger diameter or higher gauge cerclage sutures whereas a thinner blade, may be used to cut smaller diameter or lower gauge cerclage sutures. Alternatively, the recess may not contain a cutting blade and the device may serve as a hook to elevate the cerclage loop as other cutting mechanism is employed to cut the suture.

The handle 24 of the device may include a actuator 70, for controlling the retractable cutter 50. The actuator may be located on the handle to allow the device to be actuated by squeezing the handle. In another embodiment, the actuator may be a pushing element coupled to the handle or near the proximal end of the shaft that allows the user to actuate the retractable cutter 50 by pressing the pushing element with a thumb or finger. The actuator may be coupled to any portion of the handle or proximal end of the shaft that allows a user to activate the cutter when the device is inserted into the vaginal canal. The actuator should be easy to use and may be reused for multiple cuttings on the same patient or different patients. The handle may be designed so that it is easy to guide the device through the vaginal canal and the cervical mucosa and to actuate the retractable cutter with one hand. The handle should be designed so that it does not prevent clear visualization of the cervix and the cerclage. Alternatively, as shown in FIGS. 22-24, the handle 24 may include other suitable shapes to allow a user to securely grip the device during use. For example, as described previously, the shaft can have sections, e.g., with a larger cross sectional diameter portion and a smaller cross sectional diameter portion. The handle 24 may be a section of the shaft having a larger cross sectional diameter or it may be otherwise disposed at the proximal end of shaft. The handle may be disk shaped as shown in FIG. 22 or may be cylindrical in shape as shown in FIG. 23. The handle may include beveled or chamfered edges, it may be spherical or hemispherical in shape, or otherwise grip shaped as shown in FIG. 24.

A rod 72 can be coupled to the actuator and the retractable cutter. The rod may be provided inside of the shaft. The rod may have a distal portion that is coupled to the cutter and a proximal portion that is coupled to the actuator. The distal portion of the rod may be shaped on an inclined plane. Upon actuation of the actuator, the distal end of the rod may slide under the retractable cutter, thus moving the cutter into a closed position. Other suitable configurations for actuating the retractable cutter to cut the suture may also be used. For example, the distal end of the rod may be coupled to the cutter. The rod may be housed within a channel in the shaft. The channel may have a distal end and a proximal end. The distal end of the channel may form an incline. Upon actuation of the actuator, the rod may move distally through the channel to the inclined region. Then, the distal end of the rod may be elevated by the inclined region, thus moving the cutter coupled to the distal end of the rod to a closed position. The length of the rod can be adjusted to allow for the depth of the vaginal canal in addition to the handle for maneuvering of the device.

A spring system may be coupled to the actuator. Upon actuation of the cutter by the actuator, the spring system may compress to allow the actuator to actuate the cutter into the closed position. Once pressure is released from the actuator, the spring system may expand to allow the cutter to move to an open position.

A light source (not shown) may be coupled to the device to allow for visualization of the cerclage. The light source may be coupled to the blunt end 32 of the shaft. In another embodiment, the light source may be coupled to the proximal end of the shaft and illuminate the blunt end of the shaft to allow for visualization of the cerclage and the cutting of the suture. Other suitable locations for positioning the light source to allow visualization of the cerclage can also be used. For example, the light source may be on the outside of the vaginal canal and separate from the device and also allow for visualization of the cerclage.

The device may be a multi-use product or a single-use, disposable product. If multi-use, the design should allow for convenient cleaning and sterilization.

Other embodiments are within the scope of the following claims. For example, while certain materials have been described, others may be used. A particular configuration of the retractable cutter has been shown, for example, in FIGS. 5-7, but other configurations could also be used to retract the cutter after a cervical suture has been cut. Particular configurations of a stationary blade or blades are shown, for example, in FIGS. 9-24, but other configurations could also be used to cut the suture.

1. A device comprising:
a shaft sized and shaped to extend from a proximal end outside a vagina to a distal end near a cervix;
the shaft having a blunt end located at the distal end of the shaft and having a taper for allowing the blunt end to pass between a cerclage suture and cervical mucosa to move the suture away from the mucosa without cutting the mucosa;
a movable cutter at the distal end of the shaft and being movable between an open non-cutting position and a closed cutting position for cutting the suture; and
an actuator for causing the cutter to be moved from the open position to the closed position to cut the suture.

2. A device comprising:
a shaft having a length sufficient to extend from a proximal end outside a vagina to a distal end near a cervix;
the shaft having a head located at the distal end of the shaft,
the head having a blunt end,
the blunt end having a tapered area for allowing the blunt end to pass between a cerclage suture and cervical mucosa to move the cerclage suture away from the mucosa without cutting the mucosa;
a lip extending from the blunt end and defining a recess in the head to hold the cerclage suture and to prevent the cerclage suture from slipping off of the blunt end before the cerclage suture is cut,
at least one blade located in the recess that is configured to cut the cerclage suture when the cerclage suture is held in the recess and the device is moved relative to the suture, wherein the blade is stationary relative to the shaft; and
a handle at the proximal end of the shaft.

3. The device of claim 2, further comprising a speculum inserted into the vagina to hold open a vaginal canal.

4. The device of claim 2, wherein the at least one stationary blade comprises a cutting edge that is disposed substantially perpendicular to the shaft.

5. The device of claim 2, wherein the distal end of the shaft consists essentially of the head having a blunt end,
the blunt end having a tapered area for allowing the blunt end to pass between a cerclage suture and cervical mucosa to move the cerclage suture away from the mucosa without cutting the mucosa;
a lip extending from the blunt end and defining a recess in the head to hold the cerclage suture and to prevent the cerclage suture from slipping off of the blunt end before the cerclage suture is cut,
a blade located in the recess, wherein the blade is stationary relative to the shaft;
the stationary blade having a cutting edge that is disposed substantially perpendicular to the shaft and is configured to cut the cerclage suture when the cerclage suture is held in the recess and the device is moved relative to the suture.

6. The device of claim 2, wherein the at least one stationary blade comprises a cutting edge that is disposed at an obtuse angle relative to the shaft.

7. The device of claim 2, wherein the at least one stationary blade comprises a cutting edge that is disposed at an acute angle relative to the shaft.

8. The device of claim 2, further comprising at least two stationary blades located in the recess.

9. The device of claim 2, wherein the recess is defined between the lip and the shaft.

10. The device of claim 2 wherein the handle has either a cross sectional area that is different from the cross sectional area of the shaft or a cross sectional shape that is different from the cross sectional shape of the shaft.

11. The device of claim 2, further comprising a light source for visualization of the suture.

12. The device of claim 2, wherein a surface of the blunt end for passing between the cerclage suture and cervical mucosa does not include a blade.

13. A cerclage removal device comprising:
a shaft having a cross sectional area and cross sectional shape and a length sufficient to extend from a proximal end outside a vagina to a distal end near a cervix;
the shaft having a head located at the distal end of the shaft,
the head having a blunt end,
the blunt end having a tapered area for allowing the blunt end to pass between a cerclage suture and cervical mucosa to move the cerclage suture away from the mucosa without cutting the mucosa;
a lip extending from the blunt end and defining a recess in the head to hold the cerclage suture and to prevent the cerclage suture from slipping off of the blunt end before the cerclage suture is cut,
a blade disposed in the recess, the blade having a cutting edge that is substantially perpendicular to the shaft; and
a handle at the proximal end of the shaft, wherein the handle has either a cross sectional area that is different from the cross sectional area of the shaft or a cross sectional shape that is different from the cross sectional shape of the shaft.

14. The device of claim 13, wherein the recess is defined between the lip and the shaft.

15. The device of claim 13, further comprising a light source for visualization of the suture.

16. The device of claim 13, wherein a surface of the blunt end for passing between the cerclage suture and cervical mucosa does not include a blade.

17. A method comprising:
inserting a shaft having a head with a blunt end into a patient's vagina;
positioning the blunt end under a cervical suture to cause the suture to move away from a cervical mucosa such that a portion of the suture is located in a recess defined by the head and lip extending from the blunt end, wherein the outer surface of the blunt end that passes under the cervical suture does not have a cutting blade,
wherein at least one cutting blade is located within the recess and is stationary relative to the shaft;
moving the shaft with respect to the suture to cause the cutting blade to cut the suture; and
withdrawing the shaft from the patient's vagina.

18. A method of claim 17, further comprising prior to the inserting the shaft, inserting a speculum into the patient's vagina, then inserting the shaft through the speculum.

19. The method of claim 17 wherein moving the shaft comprises pulling the shaft away from the patient's vagina.

20. A device comprising:
a shaft sized and shaped to extend from a proximal end outside a vagina to a distal end near a cervix;
the shaft having a blunt end located at the distal end of the shaft and having a taper for allowing the blunt end to pass between a cerclage suture and cervical mucosa to move the suture away from the mucosa without cutting the mucosa;
a cutter at the distal end of the shaft; and
a handle at the proximal end of the shaft.

21. The device of claim 20, further comprising a speculum inserted into the vagina to hold open a vaginal canal.

22. The device of claim 20, further comprising a lip on the blunt end to hold the suture and to prevent the suture from slipping off of the blunt end of the shaft before the suture is cut.

23. The device of claim 22, further comprising a recess on the blunt end of the shaft for holding the suture.

24. The device of claim 20, further comprising a light source for visualization of the suture.

25. The device of claim 20, wherein a surface of the blunt end for passing between the cerclage suture and cervical mucosa does not include a blade.

26. A device comprising:
a shaft having a length sufficient to extend from a proximal end outside a vagina to a distal end near a cervix;
the shaft having a head located at the distal end of the shaft,
the head having a blunt end,
the blunt end having a tapered area for allowing the blunt end to pass between a cerclage suture and cervical mucosa to move the cerclage suture away from the mucosa without cutting the mucosa;
a lip extending from the blunt end and defining a recess in the head to hold the cerclage suture and to prevent the cerclage suture from slipping off of the blunt end before the cerclage suture is cut,
at least one blade configured to cut the cerclage suture when the cerclage suture is held in the recess; and
a handle at the proximal end of the shaft.

27. The device of claim 26, further comprising a speculum inserted into the vagina to hold open a vaginal canal.

28. A method comprising:
inserting a shaft having a blunt end into a patient's vagina;
positioning the blunt end under a cervical suture to cause the suture to move away from a cervical mucosa, wherein the blunt end that passes under the cervical suture does not have a cutting blade;
cutting the suture with a blade; and
withdrawing the shaft from the patient's vagina.

29. A method of claim 28, further comprising prior to the inserting the shaft, inserting a speculum into the patient's vagina, then inserting the shaft through the speculum.
